(19) United States
(12) Patent Application Publication
(10) Pub. No.: US 2007/0123518 A1
(43) Pub. Date: May 31, 2007

(54) Product designed to protentiate the therapeutic effects and to enhance the action of medicinal preparations

(76) Inventor: Oleg Illich Epshtein, Moscow (RU)

Correspondence Address: Ilya Zborovsky, 6 Schoolhouse Way, Dix Hills, NY, 11746, US

(21) Appl. No.: 10/574,524

(22) PCT Filed: Sep. 27, 2004
(86) PCT No.: PCT/RU04/00374

§ 371 Date: Mar. 31, 2006

(30) Foreign Application Priority Data
Oct. 1, 2003 (RU) 2003129126

(51) Int. Cl. A61K 31/5513; A61K 31/47

(52) U.S. Cl. 514/221; 514/310

(57) ABSTRACT

Said invention is characterised by the application of a small and mini doses of an activated medical substance in the form of an agent for potentiating curing effects, i.e enhancing action of the therapeutic dose of the same medical substance.

### FIELD OF THE INVENTION

The invention relates to medicine, namely, to pharmacology and pharmacotherapy and can be employed for enhancement of therapeutic activity pharmaceutical preparations.

### DESCRIPTION OF THE BACKGROUND ART

The use of pharmaceutical agents for potentiation of therapeutic effects—enforcement of therapeutic activity of medicinal agents is well known (see V. I. Petrov, M. D. Gaeviy, P. A. Galenko-Yaroshevsky, Fundamentals of clinical pharmacology and pharmacotherapy, M., “Alyans-V” 2002, p. 42-44). However, the difficulties with combining various ingredients and the probability of adverse effects limit the functional yield of that solution. Also known are homeopathic pharmaceutical preparations based on initial herbal raw materials (RU 2122858 C1, A 61κ 35/78, 1998; RU 2133123 C1, A 61κ 35/78, 1999; RU 2177795 C1, A 61κ 35/78, 2002) as well as activated forms of pharmaceutical agents in ultra-low doses produced by repeated consecutive dilution and shaking in accordance with homeopathic method (RU 2182492 C1, A 61κ 39/00, 2002; RU 2191601 C1, A 61κ 39/395, 2002; RU 2192882 C1, A 61κ 38/22, 2002; RU *2201255 C1, A 61κ 39/395, 27.03.2003; RU 2209083 C1, A 61κ 39/395, 27.07.2003).

These preparations are mostly intended for individualized treatment of various diseases, based on application of clinical-phenomenological principle of similarity.

### DESCRIPTION OF THE INVENTION

The invention is aimed at development of effective (universal) method to potentiate the therapeutic effects and enhance the activity of various pharmaceutical substances and preparations.

The solution of the given problem is provided by application of low or ultra-low doses of activated forms of pharmaceutical substance as the product that potentiates the therapeutic effects and enhances the activity of therapeutic doses of the same pharmaceutical substance, wherein said activated forms are obtained by repeated consecutive dilution combined with external treatment by homeopathic technology of potentiation.

Application of activated forms of pharmaceutical substance in ultra-low doses for novel indication has turned out possible due to previously unknown property of those forms to provide, on adding to the initial pharmaceutical substance, a non-summarized enhancement of therapeutic effect of the latter that is not readily apparent from well-known decisions. In itself the activated form of ultra-low doses of the pharmaceutical agent might not produce a significant therapeutic effect.

### EMBODIMENTS OF THE INVENTION

The pharmaceutical preparation aimed at potentiation of therapeutic effects—at the enhancement of activity of medicinal agent is prepared by consecutive repeated dilutions of initial medicinal substance and by simultaneous exposition of dilutions to standardized shaking until ultra-low or low doses (not containing molecules of parental substance) are obtained, for example, in accordance with homeopathic technology (see V. Shvabe, Homeopathic Pharmaceutical Agents. A Manual on Description and Preparation, Moscow, 1967, p. 12-38). At that, the concentration is proportionally reduced through consecutive dilution of 1 volumetric part of the initial substance in 9 volumetric parts (for decimal dilutions, D) or in 99 volumetric parts (for centesimal dilutions, C) of a neutral solvent until the required dose (potency) is obtained; each dilution is followed by multiple vertical mechanical shaking; for each dilution separate vessel is preferable. External treatment can also be performed in the process of dilution by sound generator and other mechanical or electromagnetic action.

The eventual activated form of ultra-low doses of the pharmaceutical preparation is added to the therapeutic dose of the same pharmaceutical agent in volumetric ratios 1:1-1:100, preferentially.

### EXAMPLE 1

Anxiolytic activity of phenazepam was studied by conflict situation model of Vogel J., 1971 in rats. Phenazepam was applied in 1) therapeutic dose, 2) activated form or 3) combination of therapeutic dose of phenazepam and activated form of the same medication. Therapeutic doses of Phenazepam (1 mg/kg) were injected intraperitoneally; activated form containing the mixture of homeopathic dilutions C12+C30+C200, was given at 2.5 ml/kg of animal's body weight intraperitoneally; combined treatment included simultaneous injections of both forms in two different syringes. The effect was evaluated after 20 minutes following to medication injections by the quantity of punished water intakes. Results are demonstrated in the table. Adverse effects related to the application of two forms of phenazepam were virtually absent.

TABLE
The impact of phenazepam in therapeutic doses, in ultra-low doses
of activated form and of their combination on the number of punished
water intakes by rats in conflict situation.

| Groups of animals | Dose | Number of punished water intakes |
|---|---|---|
| Control | | 177.75 ± 43.02 |
| Phenazepam | 1 mg/kg | 415.67 ± 113.96 * |
| Activated form | 2.5 ml/kg | 260.67 ± 38.21 |
| in ultra-low doses | of body weight | |
| Combination of two | 2.5 ml/kg + | 1279.33 ± 82.28 ** |
| forms | 1 mg/kg | |

* Significant difference from control,
** Significant differences from the group F, p < 0.05.

The gained data demonstrate that phenazepam (1 mg/kg) possesses anxiolytic activity. Anxiolytic effect of activated form of phenazepam in ultra-low doses proves distinct from that of control though the difference lacked statistical significance. Combined administration of phenazepam and of its activated form in ultra-low doses produces anxiolytic effect exceeding that of phenazepam 3-fold and summarized effects of both forms on separate injections in 1.9 times. Thus, injection of ultra-low doses of activated form of the medication together with therapeutic dose of phenazepam provides potentiation of specific pharmacological activity of phenazepam in therapeutic dose.

### EXAMPLE 2

Benzodiazepines represent reference medications for the treatment of anxious states. However, application of benzodiazepines is frequently associated with adverse effects that require dose reduction to minimal effective doses. Investigation of impacts induced by ultra-low doses of activated form of diazepam on the anxiolytic activity of therapeutic doses of diazepam was accomplished in 20 patients suffering from generalized anxiety syndrome characterized with the anxiety level consistent with at least 20 grades of Hamilton's scale of anxiety. Patients received tablets containing activated form of diazepam in ultra-low doses (homeopathic dilution C200, equivalent concentration—10⁻⁴⁰⁰M mass concentration)—1 tablet 4 times a day for 14 days. Thereafter, beginning from 15^{th} to 30^{th} day of therapy patients were took per oral diazepam at the dose of 2 mg qid day (daily dose—8 mg) followed by administration of ultra-low doses of activated form of the very medication 4 tablets per day for 7 days and by combined treatment implicating simultaneous intake of activated form (4 tablets a day) and diazepam (8 mg per day) for 14 days. Anxiety level was evaluated by means of Hamilton's scale at baseline and after each stage of therapy. Results are demonstrated in table.

TABLE
Influences of activated form of diazepam either in ultra-low dose
or in therapeutic dose (8 mg/day) and of combined receipt of both
forms on the level of anxiety in generalized anxious states.

| | Overall level of anxiety in |
|---|---|
| | accordance with Hamilton's scale, |
| | grades (improvement versus |
| TERMS OF TESTING | baseline, %) |
| Baseline | 27.7 ± 2.6 |
| After activated form of diazepam | 23.5 ± 3.1 (15%) |
| in ultra-low doses-2 weeks | |
| After diazepam 8 mg/day | 16.2 ± 2.5 * (26%) |
| After activated form of diazepam | 20.5 ± 3.4 |
| in ultra-low doses-1 week | |
| After combined application of | 10.3 ± 1.8 * (63%) |
| both forms | |

* Significant difference from previous stage, p < 0.05.

Dynamic changes in the anxiety level have demonstrated that activated form of diazepam in ultra-low doses did not exert statistically significant anxiolytic effects (p. 2, table 4). Administration of suboptimal therapeutic dose of diazepam does not yield achievement of pronounced decline of anxiety (p. 3 of table). Combined application of both forms contributes to the therapeutic effect (p. 5) that does not yield to the impacts of higher doses of diazepam (15 mg/day—according to reference data) and does not entail marked adverse effects. Administration of activated form of diazepam in ultra-low doses together with diazepam results in potentiation of specific pharmacological activity of diazepam.

### EXAMPLE 3

Rat model of adjuvant arthritis induced by single subplantar injection of 100 μl of complete Freund's adjuvant was applied for evaluation of anti-inflammatory activity of glucocorticosteroid agent—prednisone in following treatment choices: a) therapeutic dose, b) activated (potentiated) form (ultra-low dose), c) combined treatment with therapeutic dose and activated form. Soon after the injection of complete Freund's adjuvant animals (10 rats per group) received prednisone in therapeutic dose, in activated form or their combination (control—distilled water)—for 14 days.

2 mg/kg prednisone (water solution) was given into the stomach; activated form of prednisone in ultra-low doses—mixture of homeopathic dilutions D24+D60 was injected in the form of water solution at the dose of 2.5mg/kg of animal's body weight into stomach; both forms mixed at 10:1 ratio were injected at one time during combined treatment.

Severity of inflammatory damage to each extremity (0 to 4) and overall index of arthritis severity (0 to 16) were assessed once per 1-2 days. Functional disturbances, erythema, edema and deformation of extremities were taken into account. Anti-inflammatory efficacy of the medications was evaluated in accordance with reduction of overall index of arthritis severity by the 2-3^{rd} and 14^{th} days of treatment. Results are demonstrated in the table.

TABLE
Anti-inflammatory activity of prednisone in therapeutic dose,
in ultra-low dose and in the form of their combination.

| | Arthritis severity index by | Arthritis severity |
|---|---|---|
| | the 2-3^{rd} day of therapy, | index by 14^{th} day |
| Group | grades | of therapy, grades |
| CONTROL | 3.4 ± 0.4 | 9.2 ± 1.1 |
| Prednisone 2 mg/kg | 2.1 ± 0.4 * | 5.4 ± 1.7 * |
| Activated form of | 2.9 ± 0.3 | 8.7 ± 1.5 |
| prednisone in | | |
| ultra-low doses | | |
| Combined treatment | 1.2 ± 0.3 *, # | 3.3 ± 1.2 *, # |

* Significant differences from control,
# Significant difference from the prednisone group 2 mg/kg, p < 0.05.

Gained data demonstrate that anti-inflammatory efficacy of activated form of prednisone in ultra-low doses is not of statistical significance. At the same time activated form of preparation yields considerable potentiation of activity of suboptimal therapeutic dose of prednisone given at earlier stage of inflammatory reaction as well as during “second tide” of immune inflammation.

### EXAMPLE 4

Twenty patients suffering from definite rheumatoid arthritis (oligoarthritis) necessitating peri- or intra-articular injections of hydrocortisone were included in the clinical trial of influences of activated form (ultra-low doses) of anti-inflammatory agent—hydrocortisone on the therapeutic activity of hydrocortisone in relation with arthritis manifestations. At baseline all patients required injections of 20-25 mg hydrocortisone 2-3 times a week (up on request). Over 8 weeks patients received activated form of hydrocortisone in ultra-low doses per orally—1 tablet for dissolution in the mouth (activated form—mixture of homeopathic dilutions C12+C30+C200, equivalent concentration 10⁻²⁴M mass concentration) together with injections. Impacts of activated form were evaluated in accordance with duration of periods, when patients were independent from hydrocortisone injections. Research results are shown in the table.

TABLE
The impact of activated form, of hydrocortisone on the duration
of therapeutic effect of hydrocortisone in therapeutic doses.

| | Duration effects of injections |
|---|---|
| | (time lag between injections |
| Treatment regimen | “upon request”), days |
| 1. Hydrocortisone 20-25 mg | 2.8 ± 0.3 |
| 2. Hydrocortisone 20-25 mg + activated | 6.3 ± 0.5 * |
| form of hydrocortisone in ultra-low | |
| doses given per os | |

* Significant differences from proper control, p < 0.05.

Thus, activated form of hydrocortisone in ultra-low doses potentiates anti-inflammatory activity of therapeutic doses hydrocortisone.

### EXAMPLE 5

Present investigation was aimed at experimental study of effects of activated form of cyclophosphamide in ultra-low doses on anti-tumor and anti-metastatic activity of therapeutic doses of cyclophosphamide. C57B1/6 line mice of both genders with 18-25 g of body weight were applied in experimental trial. Lung carcinoma of Louise that is known to metastasize in hematogenous way (3LL), lung carcinoma-67 (LC-67) and melanoma B-16 (B-16) were transplanted intramuscularly. Cyclophosphamide at 125 mg/kg dose was injected once intraperitoneally on 11^{th} (3LL), 12^{th} (LC-67) and 16^{th} (B-16) days following tumor transplantation. Preparation potentiated by homeopathic technology that contained mixture of homeopathic dilutions of cyclophosphamide C12+C30+200 (equivalent concentrations were 10⁻²⁴, 10⁻⁶⁰ and 10⁻⁴⁰⁰M mass concentrations, respectively)—activated form of cyclophosphamide—was given at 0.3ml dose per mouse into stomach for 9-10 days beginning at an hour following to injection of cytostatic agent. Proportion of experimental group animals was subjected to intraperitoneal injections of physiological solution instead of therapeutic dose of cyclophosphamide. Control group mice received potentiated water into stomach. Assessment of effectiveness of therapeutic influences was performed by 19^{th} (3LL), 21^{st} (LC-67) and 27^{th} (B-16) days of experiment by tumor mass evaluation (difference between the weights of healthy and tumorous paws) and by estimation of tumor growth inhibition—relative diminution of tumor mass compared to control group. Intensity of tumor dissemination was evaluated using mean quantity and average area of metastatic nodes per mouse in a group as well as incidence of metastatic tumor in percents (number of animals suffering from metastatic disease in relation with the total quantity of mice in the given group).

Course injections of activated form of cyclophosphamide in ultra-low doses after injections of physiological solution did not exert anti-tumor and anti-metastatic activities (in comparison with control). Course injections of activated form of cyclophosphamide in ultra-low doses accomplished against a background of 3LL chemotherapy did not yield significant changes in the weight of primary tumor node though resulted in significant enhancement of anti-metastatic effect of therapeutic doses of cyclophosphamide corroborated by the absence of visible lung metastasis in experimental group animals. Application of ultra-low doses of activated form of cyclophosphamide together with cytostatic therapy of LC-67 the number and total area of metastasis reduced in 3.8 and 12.5 times, respectively. Metastatic affection of lungs was observed in only 25% of mice. At the same time, injection of activated form of cyclophosphamide in ultra-low doses to mice suffering from LC-67 produced considerable enhancement of inhibitory effects of cytostatic agent on the primary tumor node. Combination of activated form with therapeutic dose of cyclophosphamide contributed to the demonstration of inhibitory impacts of preparation on advancement of melanoma B-16 dissemination. Thus, inclusion of activated form of cyclophosphamide in the treatment scheme led to significant reduction of corresponding parameter in a group of combination therapy. Thus, capacity of activated form of cyclophosphamide in ultra-low doses to potentiate anti-tumor and anti-metastatic activity of therapeutic doses of the cytostatic agent was shown in three experimental tumor models. It is remarkable that observed enhancement of anti-tumor and anti-metastatic effects of chemotherapy was not accompanied with toxicity aggravation.

### EXAMPLE 6

Impacts of activated form of widely studied in psychopharmacology medication—of ethanol on the anxiolytic effect of ethanol was evaluated using experimental model of conflict situation by Vogel J., 1971 (conflict of drinking motivation and painful electric irritation in conditions of water deprivation) in rats. Ethanol (10% water solution), water solution of activated form of ethanol in ultra-low doses (homeopathic dilution C1000, equivalent concentration—10⁻²⁰⁰⁰ mass concentrations) or mixture of 10% ethanol and activated form of ethanol in 10:1 volumetric ratio was brought in the drinking bowl of chamber for investigation of punished drinking behavior. Anxiolytic effect was evaluated in accordance with the number of punished water intakes. Results are demonstrated in the table.

| Group animals | Number of punished water intakes |
|---|---|
| 1. Control | 170.5 ± 38.2 |
| 2. Ethanol 10% | 317.2 ± 41.0 * |
| 3. Activated form of ethanol in ultra- | 205.3 ± 53.6 |
| low doses | |
| 4. Ethanol 10% + activated form | 396.7 ± 28.1 *, # |

* Significant difference from control,
** Significant difference from group-2, p < 0.05

Thus, activated form of ethanol in ultra-low doses did not exert statistically significant anxiolytic activity in conditions of conflict situation though it potentiates anxiolytic effect of 10% ethanol solution.

### EXAMPLE 7

Influences of activated form of morphine in ultra-low doses on analgesic effect of morphine were evaluated using a test of withdrawal of extremities in response to electric irritation in rats. Rats included in different experimental groups underwent single intraperitoneal injection of: a) morphine in therapeutic dose (5 mg/kg); b) activated form of morphine in ultra-low doses (homeopathic dilution C200, equivalent concentration 10⁻⁴⁰⁰ M mass concentrations)—0.5 ml water solution; c) therapeutic dose of morphine and activated form of preparation at one time (in one syringe at 5:1 volumetric ratio). Distilled water served the control. Thresholds of nociceptive responses were registered at baseline and after 30 minutes following to medication injection. Analgesic effect was assessed in accordance with elevation of nociceptive response threshold compared to the baseline data. Results are demonstrated in the table.

| | Threshold of nociceptive | |
|---|---|---|
| | response, volt | |
| | Prior to | After |
| Group of animals | injection | injection |
| 1. Control | 30.7 ± 2.5 | 31.0 ± 3.0 |
| 2. Activated form of morphine in | 31.2 ± 2.1 | 27.9 ± 2.9 |
| ultra-low doses | | |
| 3. Morphine 5 mg/kg | 29.7 ± 2.6 | 38.2 ± 2.8 * |
| 4. Injection of morphine 5 mg/kg + | 31.7 ± 2.8 | 44.5 ± 2.1 *, # |
| activated in ultra-low doses | | |

* Significant difference from baseline values,
# Significant difference from group-3, p < 0.05

Thus, single injection of activated form of morphine in ultra-low doses did not take statistically significant analgesic activity though it potentiated anesthetic effect of single injection of morphine at 5 mg/kg dose.

1. The use of an activated form of a pharmaceutical substance in low or ultra-low dose as a product that potentiates the therapeutic effects and enhances the action of the same pharmaceutical preparation in therapeutic dose.
